(19) United States
(12) Patent Application Publication
(10) Pub. No.: US 2022/0000458 A1
(43) Pub. Date: Jan. 6, 2022

(54) Reusable Biopsy Device And Related Systems And Methods

(71) Applicant: ReCore Medical Inc., New Haven, CT (US)

(72) Inventors: Walter Gottlieb Bircher, Richmond, VA (US); Marley Windham-Herman, Rancho Mirage, CA (US)

(21) Appl. No.: 17/368,696

(22) Filed: Jul. 6, 2021

Related U.S. Application Data
(60) Provisional application No. 63/048,233, filed on Jul. 6, 2020.

(51) Int. Cl. A61B 10/02; A61B 17/32

(52) U.S. Cl. CPC A61B 10/0275; A61B 2017/0023; A61B 17/32

(57) ABSTRACT

Reusable core needle biopsy devices having an energy storage and firing mechanism comprising one or more flexure components and a disposable biopsy needle. The energy storage and firing mechanism and the biopsy needle can move between a retracted (or tensioned) position and an extended position such that a user can retract device and then actuate it to drive the needle into the target tissue.

### CROSS-REFERENCE TO RELATED APPLICATION(S)

This application claims the benefit under 35 U.S.C. § 119(e) to U.S. Provisional Application 63/048,233, filed Jul. 6, 2020 and entitled “Reusable Biopsy Device,” which is hereby incorporated herein by reference in its entirety.

### FIELD

The various embodiments herein relate to medical devices, and more specifically to biopsy devices for collecting tissue from a patient.

### BACKGROUND

Interventional radiology (“IR”) is a medical specialty that focuses on minimally invasive procedures such as stroke treatment, stent placement, and diagnostic biopsies. Core needle biopsies (“CNB”), critical to cancer diagnoses, are one of the most common IR procedures and, in developed markets, represent a high-volume procedure with high iterative and per-procedure costs.

Disadvantages of known disposable biopsy devices include high iterative costs and large stocking volume, while disadvantages of known reusable biopsy devices include the fact that they are constructed primarily of metal and therefore present prohibitive upfront costs and costly servicing requirements. In addition, most known biopsy devices rely on some type of tensionable spring for actuation, which requires substantial volume and thus increases the size of the biopsy devices. In addition, any attempt to increase the force of the actuation requires either an increase in the size of the spring or the number of springs, thereby requiring an additional increase in the size of the device.

There is a need in the art for an improved biopsy device and related systems and methods.

### BRIEF SUMMARY

Discussed herein are various embodiments of biopsy devices for use in collecting tissue samples from patients.

In Example 1, a biopsy device comprises a body and a biopsy needle extending from the body. The biopsy needle comprises an outer cannula and an inner needle slidably disposed within the outer cannula. The biopsy device further comprises an actuation mechanism disposed within the body and operably coupled to the outer cannula such that outer cannula is axially constrained in relation to the actuation mechanism, the actuation mechanism comprising at least one tensionable member, wherein the at least one tensionable member is moveable between a first member position and a second member position, and an actuation handle operably coupled to the inner needle such that the inner needle is axially constrained in relation to the actuation mechanism, wherein the outer cannula is slidably coupled to the actuation handle, wherein the actuation handle is moveable between a first handle position, a second handle position, and an intermediate handle position, wherein movement of the handle from the intermediate handle position toward the first handle position causes the actuation mechanism to urge the outer cannula distally.

Example 2 relates to the biopsy device according to Example 1, wherein when the actuation handle is in the first handle position and the at least one tensionable member is in the first member position, the outer cannula and the inner needle are disposed in an extended position.

Example 3 relates to the biopsy device according to Example 1, wherein when the actuation handle is in the second handle position and the at least one tensionable member is in the second member position, the outer cannula and the inner needle are disposed in a retracted position.

Example 4 relates to the biopsy device according to Example 1, wherein when the actuation handle is in the intermediate handle position and the at least one tensionable member is in the second member position, the outer cannula and the inner needle are disposed in a tissue collection position, wherein a distal end of the inner needle extends from the outer cannula such that a tissue collection channel on the inner needle is exposed.

Example 5 relates to the biopsy device according to Example 1, wherein the outer cannula is urged distally at a speed ranging from about 1.5 m/s to about 15 m/s and a force ranging from about 15 N to about 50 N.

Example 6 relates to the biopsy device according to Example 1, wherein the at least one tensionable member is attached at a first end to a first side wall of the body and at a second end to a second side wall, wherein the at least one tensionable member is spaced from a top wall and a bottom wall of the body.

In Example 7, a biopsy device comprises a body and a biopsy needle extending from a distal end of the body, the biopsy needle comprising an outer cannula, and an inner needle slidably disposed within the outer cannula. The biopsy device further comprises an actuation handle extending from a proximal end of the body and coupled to the inner needle, the actuation handle comprising an elongate handle body moveably disposed at least partially within the body and a piston cavity defined within the elongate body, wherein the actuation handle is moveable between a distal handle position, a proximal handle position, and an intermediate handle position. The biopsy device also comprises an actuation mechanism disposed within the body, the actuation mechanism comprising a cannula piston moveably disposed within the piston cavity and coupled to the outer cannula and at least one tensionable member coupled to the cannula piston, wherein the tensionable member is movable between a distal member position and a proximal member position.

Example 8 relates to the biopsy device according to Example 7, wherein movement of the handle from the intermediate handle position toward the distal handle position causes the cannula piston to move distally, whereby the at least one tensionable member becomes tensioned and thereby urges the outer cannula distally.

Example 9 relates to the biopsy device according to Example 8, wherein the outer cannula is urged distally at a speed ranging from about 1.5 m/s to about 15 m/s and a force ranging from about 15 N to about 50 N.

Example 10 relates to the biopsy device according to Example 7, wherein the outer cannula comprises a cannula coupling body at a proximal end of the outer cannula, and the cannula piston comprises a cannula cavity defined with the cannula piston, wherein the cannula cavity is sized and shaped to receive the cannula coupling body.

Example 11 relates to the biopsy device according to Example 7, wherein the inner needle comprises a needle coupling body at a proximal end of the inner needle, and the actuation handle comprises a needle cavity defined within elongate handle body, wherein the needle cavity is sized and shaped to receive the needle coupling body.

Example 12 relates to the biopsy device according to Example 7, wherein the at least one tensionable member comprises a at least one distal tensionable member and at least one proximal tensionable member.

Example 13 relates to the biopsy device according to Example 12, wherein the at least one distal tensionable member comprises at least two distal tensionable members coupled together and the at least one proximal tensionable member comprises at least two proximal tensionable members coupled together.

Example 14 relates to the biopsy device according to Example 7, wherein the at least one tensionable member is attached at a first end to a first side wall of the body and at a second end to a second side wall of the body, wherein the at least one tensionable member is spaced from a top wall and a bottom wall of the body such that the at least one tensionable member is not in contact with the top wall or the bottom wall.

Example 15 relates to the biopsy device according to Example 7, wherein the cannula piston is spaced from a top wall of the piston cavity and from a bottom wall of the piston cavity such that the cannula piston is not in contact with the top wall of the piston cavity or the bottom wall of the piston cavity.

Example 16 relates to the biopsy device according to Example 7, wherein the at least one tensionable member is a bistable tensionable member, wherein the at least one tensionable member is untensioned in both the distal member position and the proximal member position.

Example 17 relates to the biopsy device according to Example 7, further comprising an elongate biopsy needle opening defined in a top portion of the body, wherein the biopsy needle opening is sized and shaped to allow a biopsy needle to pass through the biopsy needle opening laterally.

In Example 18, a method of collecting tissue from a patient comprises retracting an actuation handle of a biopsy device into a retracted position, the biopsy device comprising a body and a biopsy needle extending from the body, the biopsy needle comprising an outer cannula and an inner needle slidably disposed within the outer cannula. The biopsy device further comprises an actuation mechanism disposed within the body and operably coupled to the outer cannula such that outer cannula is axially constrained in relation to the actuation mechanism, the actuation mechanism comprising at least one tensionable member disposed in a proximal position, and the actuation handle operably coupled to the inner needle such that the inner needle is axially constrained in relation to the actuation mechanism, wherein the outer cannula is slidably coupled to the actuation handle. The method further comprises inserting the biopsy needle into a patient such that a distal tip of the biopsy needle is disposed adjacent to a target tissue, urging the actuation handle distally into a tissue collection position, whereby the inner needle is urged distally such that a tissue collection channel on the inner needle extends out of the outer cannula, and urging the actuation handle distally from the tissue collection position such that the at least one tensionable member becomes tensioned and urges the outer cannula distally over the tissue collection channel, thereby severing tissue disposed in the tissue collection channel.

Example 19 relates to the method according to Example 17, wherein the urging the actuation handle distally into a tissue collection position does not move the actuation mechanism.

Example 20 relates to the method according to Example 17, wherein the urging the actuation handle distally from the tissue collection position causes the actuation mechanism to be urged distally.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a perspective view of a biopsy device with a handle in a first position, according to one embodiment.

FIG. 1B is a perspective view of the biopsy device of FIG. 1A with the handle in a second position, according to one embodiment.

FIG. 10 is a perspective view of the biopsy device of FIG. 1A with the handle in a third or intermediate position, according to one embodiment.

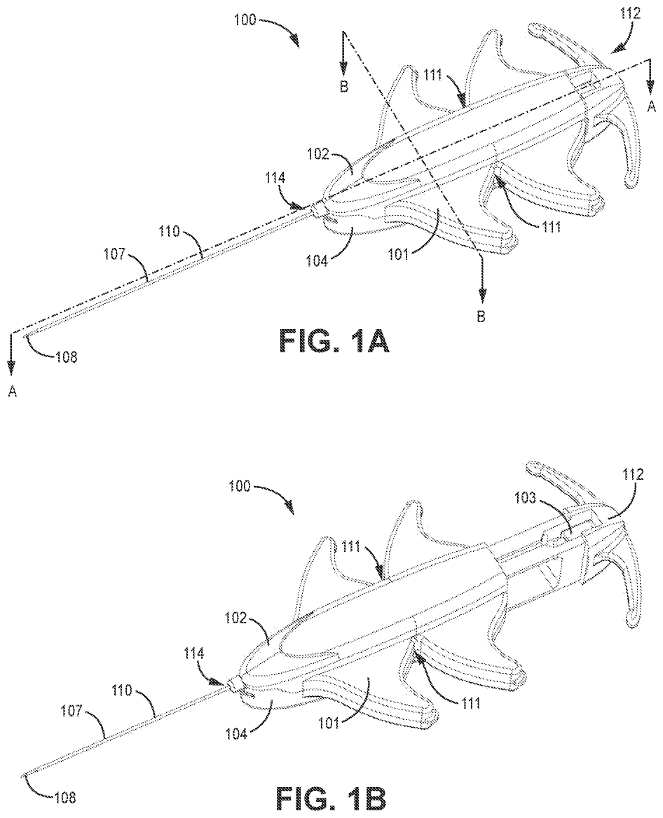

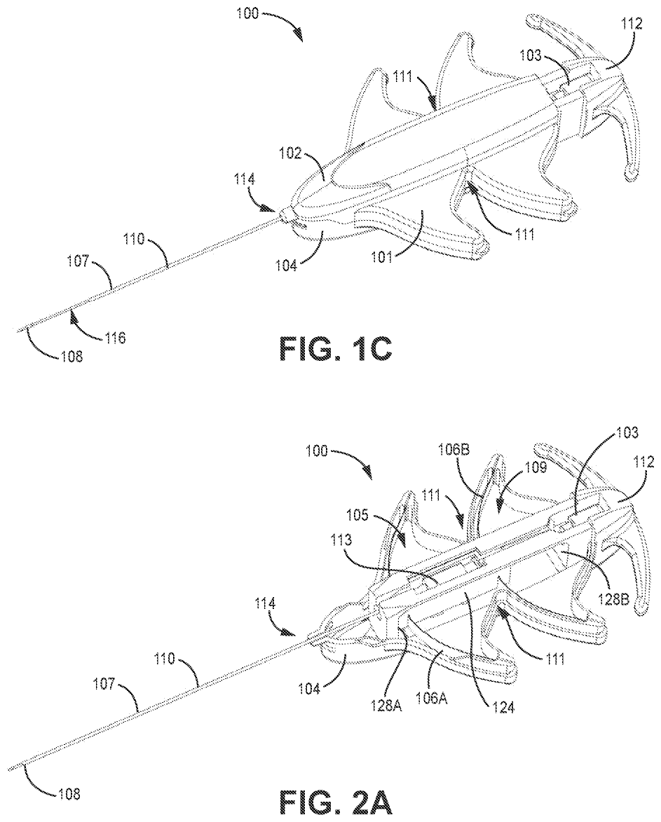

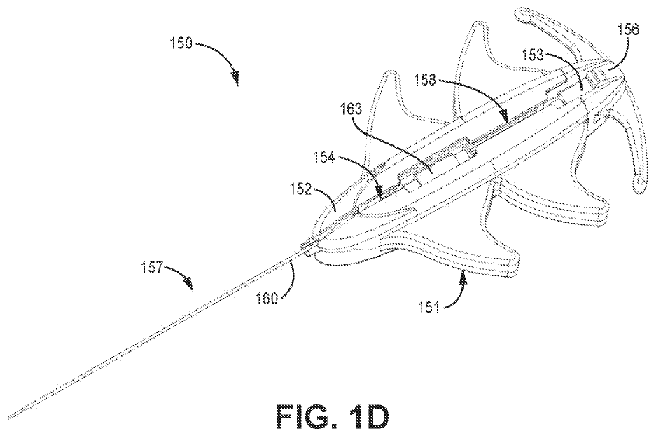

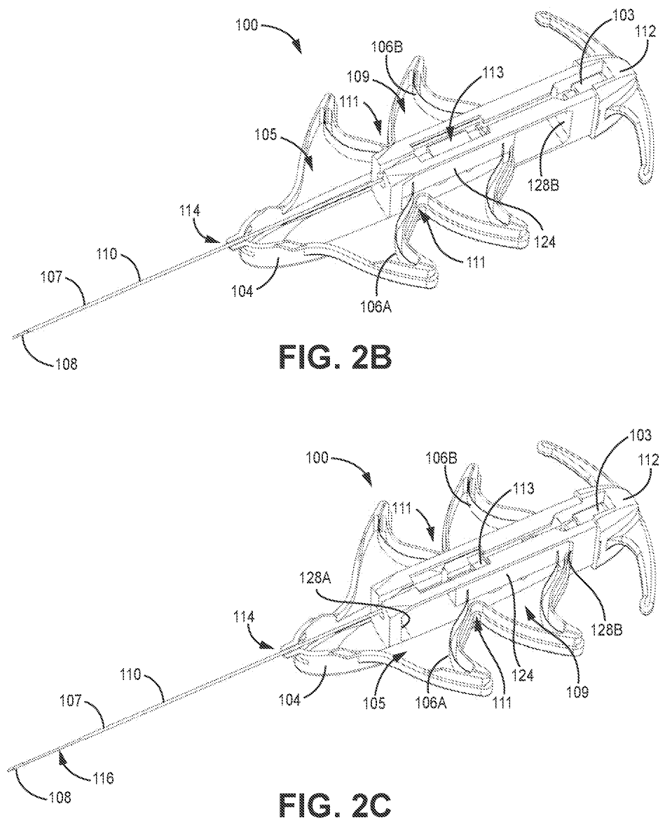

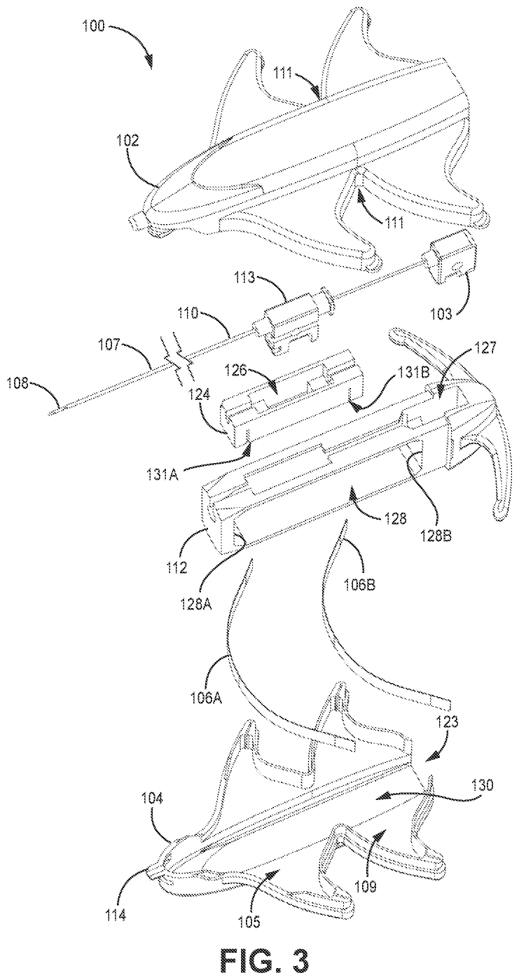

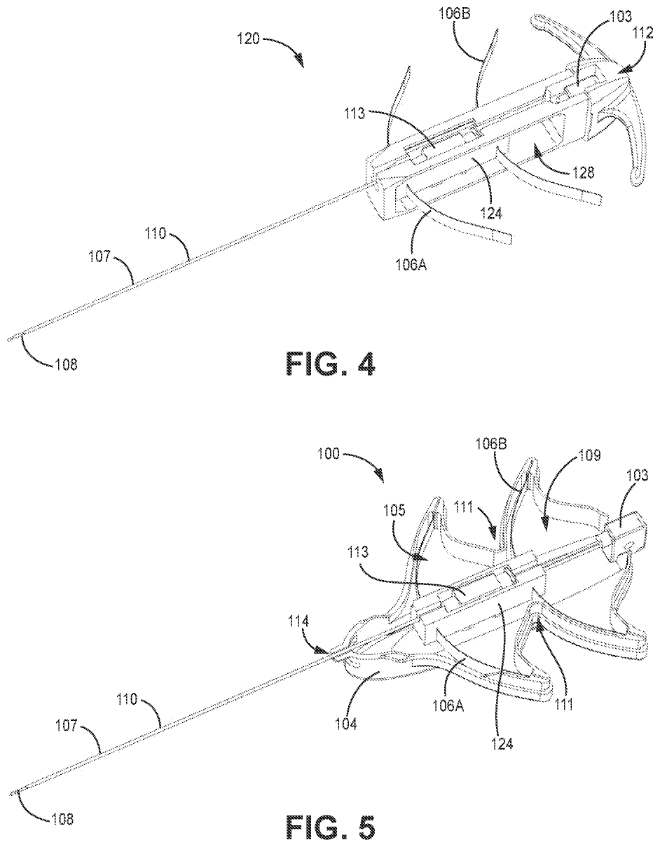

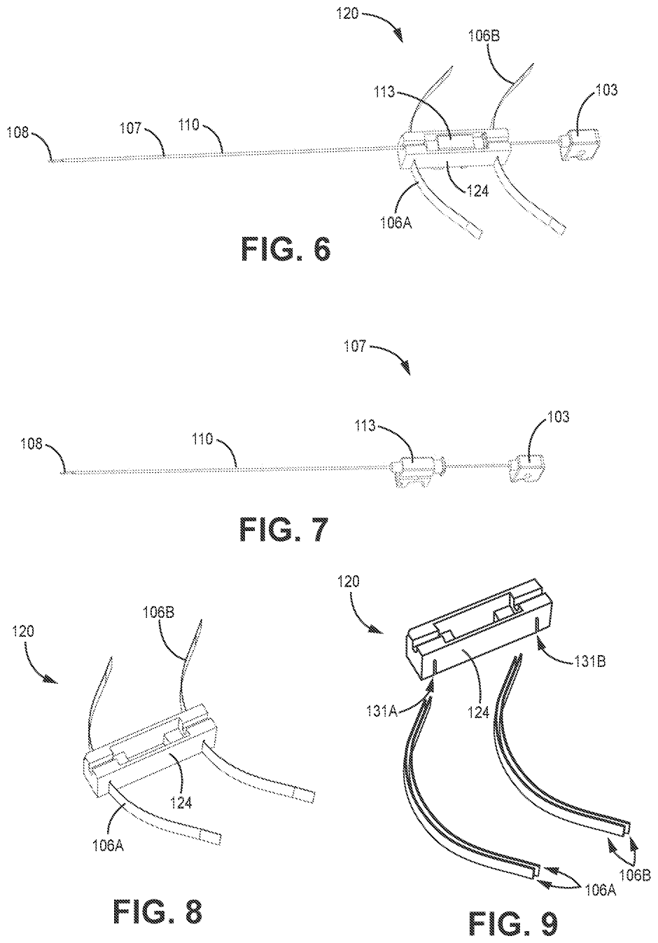

FIG. 1D is a perspective view of another biopsy device with an opening defined in the top shell, according to one embodiment.

FIG. 2A is a perspective view of the biopsy device of FIG. 1A with a top shell removed and with the handle in the first position, according to one embodiment.

FIG. 2B is a perspective view of the biopsy device of FIG. 1A with the top shell removed and with the handle in the second position, according to one embodiment.

FIG. 2C is a perspective view of the biopsy device of FIG. 1A with the top shell removed and with the handle in the third or intermediate position, according to one embodiment.

FIG. 3 depicts an exploded view of the biopsy device of FIG. 1A, according to one embodiment.

FIG. 4 is a perspective view of the biopsy needle, actuation mechanism, and handle of the biopsy device of FIG. 1A, according to one embodiment.

FIG. 5 is a perspective view of the biopsy device of FIG. 1A with the top shell removed and the biopsy needle and actuation mechanism visible, according to one embodiment.

FIG. 6 is a perspective view of the biopsy needle and actuation mechanism of the biopsy device of FIG. 1A, according to one embodiment.

FIG. 7 is a perspective view of the biopsy needle of the biopsy device of FIG. 1A, according to one embodiment.

FIG. 8 is a perspective view of the actuation mechanism of the biopsy device of FIG. 1A, according to one embodiment.

FIG. 9 is an exploded view of an actuation mechanism of the biopsy device of FIG. 1A with stacked flexure components, according to one embodiment.

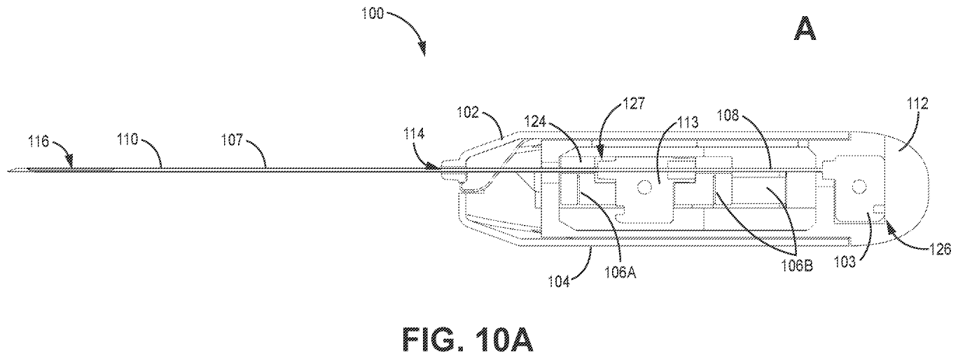

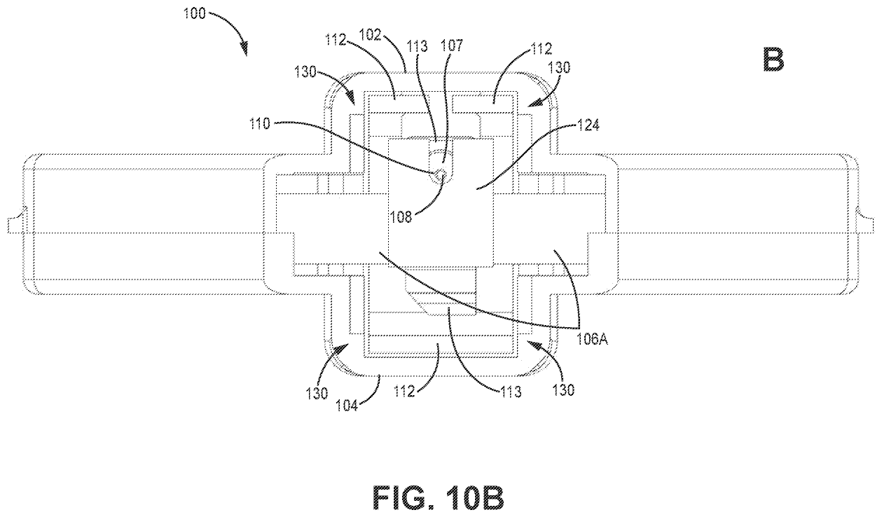

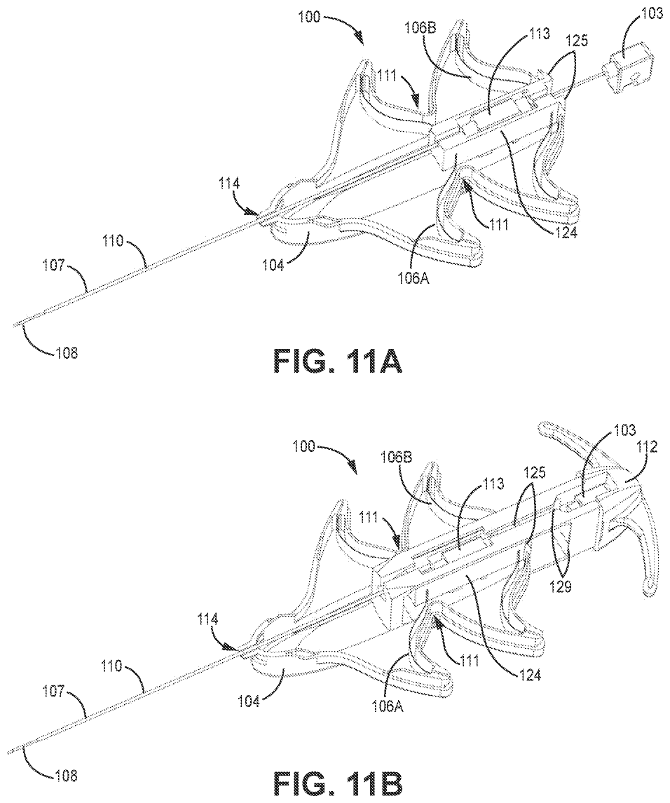

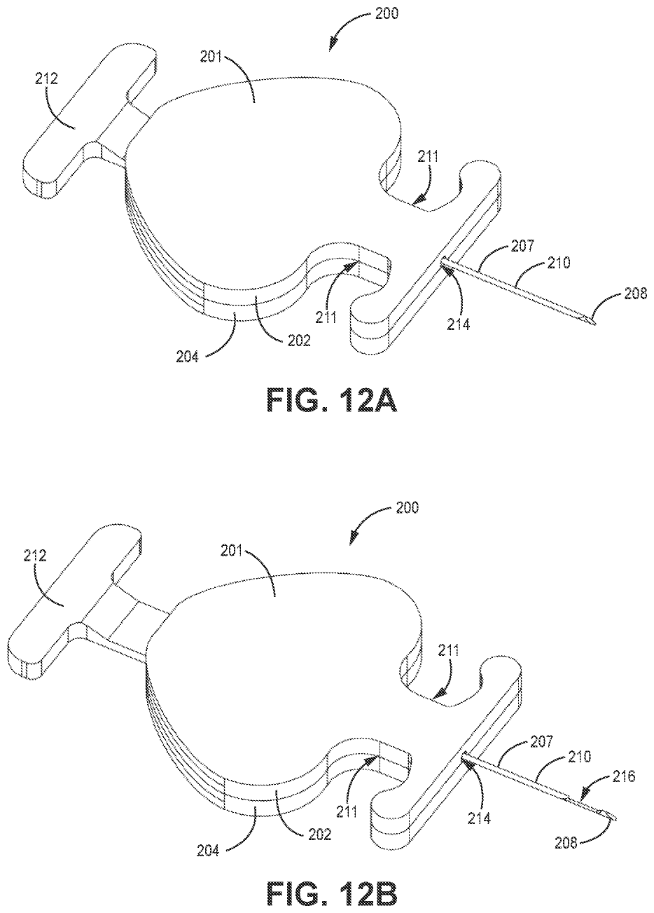

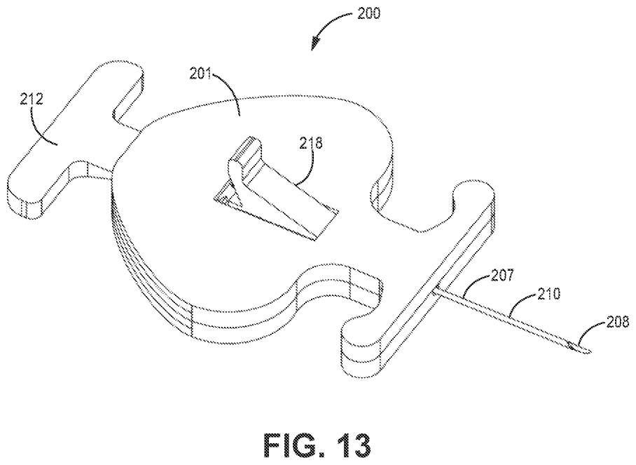

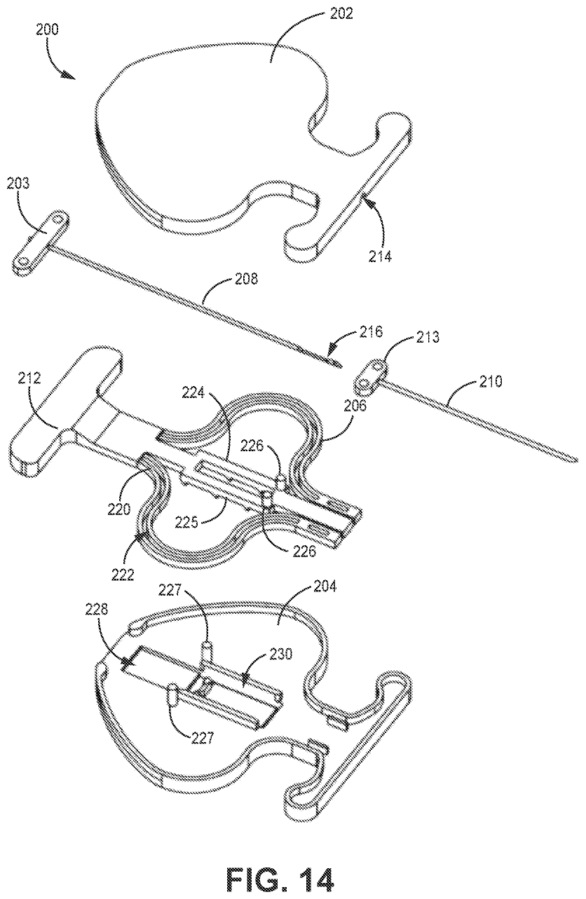

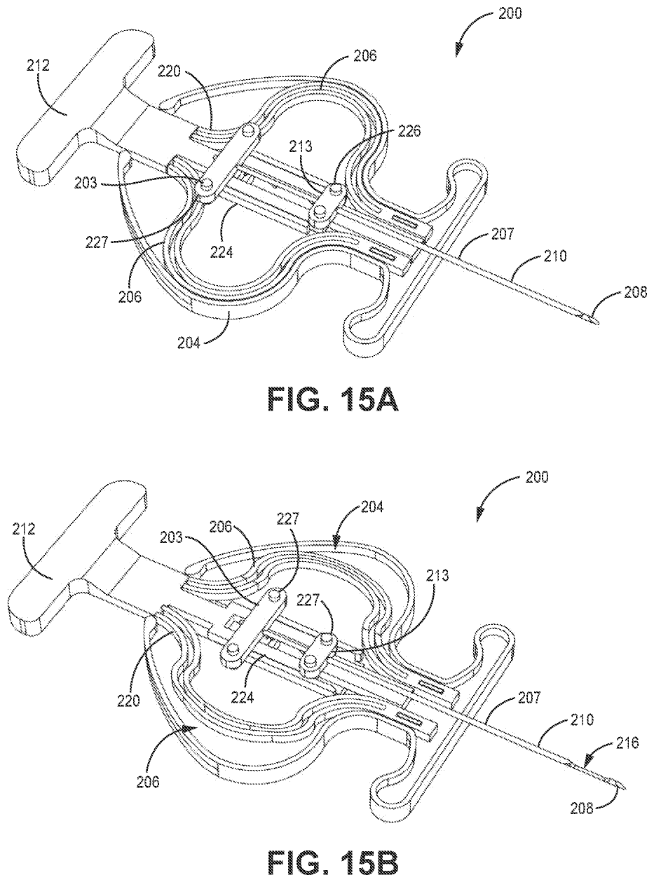

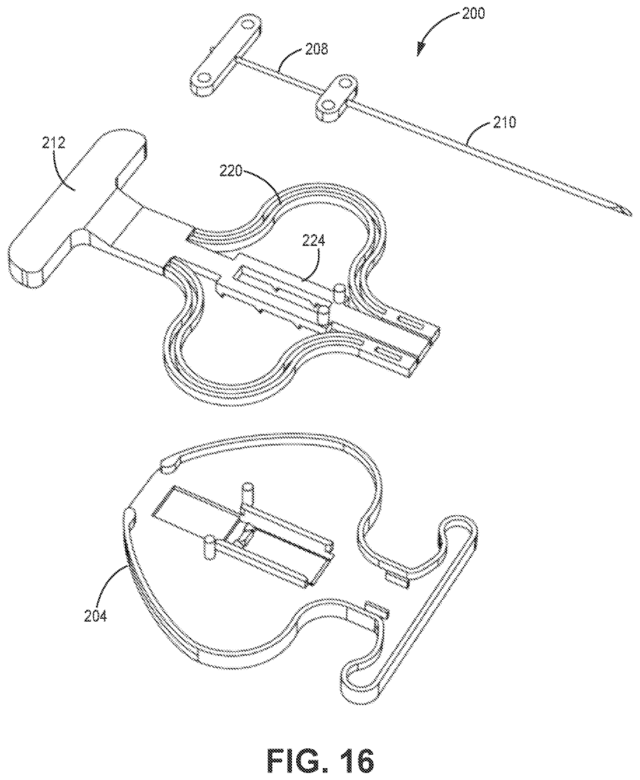

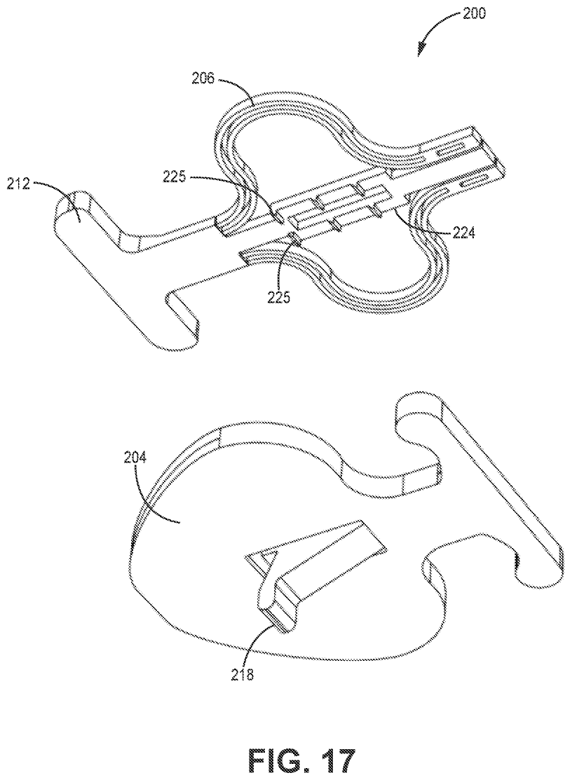

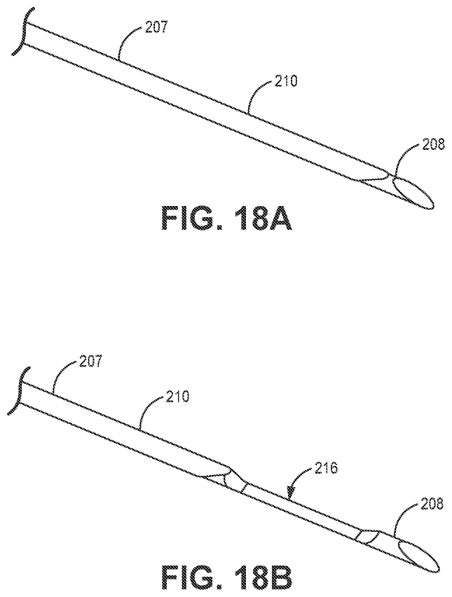

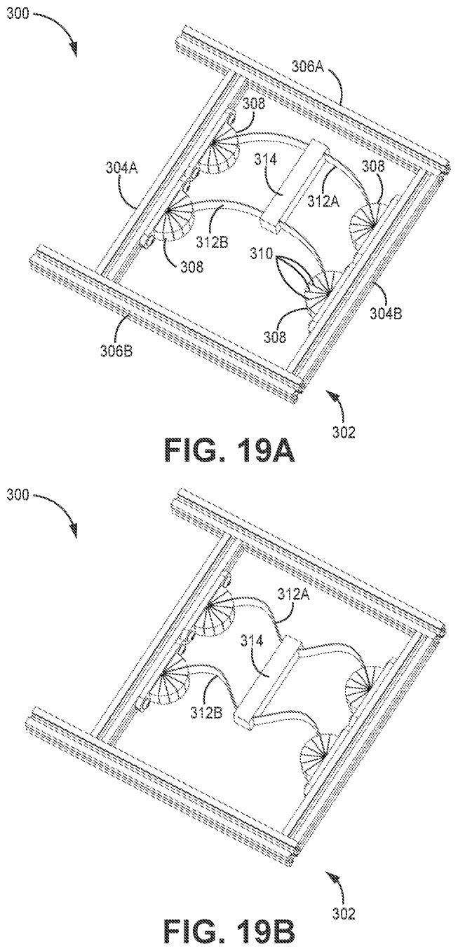

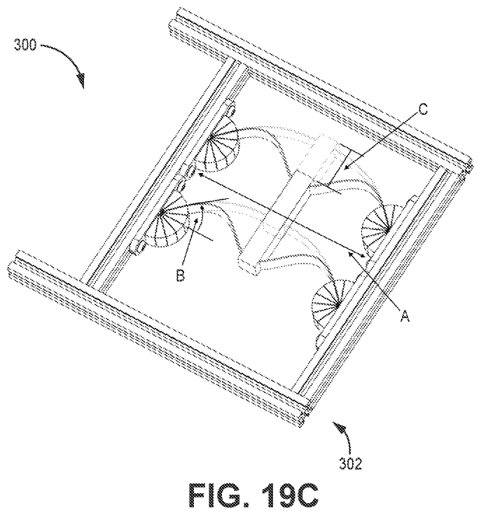

FIG. 10A is a cross-sectional side view of the biopsy device along line A of FIG. 1A, according to one embodiment.

FIG. 10B is a cross-sectional side view of the biopsy device along line B of FIG. 1A, according to one embodiment.

FIG. 11A is a perspective view of a biopsy device with a top shell removed, according to another embodiment.

FIG. 11B is another perspective view of the biopsy device of FIG. 11A with the top shell removed, according to one embodiment.

FIG. 12A is a perspective view of a biopsy device with the handle in a first position, in accordance with one embodiment.

FIG. 12B shows a perspective view of the biopsy device of FIG. 12A with the handle in a second position, in accordance with one embodiment.

FIG. 13 shows a perspective view of the underside of the reusable biopsy device of FIG. 12A with a trigger in a first position, in accordance with one embodiment.

FIG. 14 shows an exploded view of the biopsy device of FIG. 12A, in accordance with one embodiment.

FIG. 15A shows a perspective view of the biopsy device of FIG. 12A with the top half body shell removed and the device in the first position, in accordance with one embodiment.

FIG. 15B shows a perspective view of the biopsy device of FIG. 12A with the top half body shell removed and the device in the second position, in accordance with one embodiment.

FIG. 16 shows a partially exploded top view of the biopsy device of FIG. 12A, in accordance with one embodiment.

FIG. 17 shows a partially exploded underside view of the biopsy device of FIG. 12A, in accordance with one embodiment.

FIG. 18A shows a perspective view of a needle and cannula of a biopsy device, wherein the shelf of the needle is covered by the cannula, in accordance with one embodiment.

FIG. 18B shows a perspective view of the needle and cannula of FIG. 18A, wherein the shelf of the needle is exposed, in accordance with one embodiment.

FIG. 19A shows a perspective view of testing jig, according to one embodiment.

FIG. 19B shows a perspective view of the testing jig of FIG. 19A in which the tensionable components being tested are disposed in a tensioned position, according to one embodiment.

FIG. 19C shows a perspective view of the testing jig of FIG. 19A with a comparison of the tensionable components in an untensioned position and a tensioned position, according to one embodiment.

### DETAILED DESCRIPTION

The various embodiments herein relate to a biopsy device, which, according to certain implementations, can be reusable. The device, according to certain implementations, can include a unique actuator that utilizes energy storage. Other embodiments can have a disposable biopsy needle.

The various configurations herein can include a device that can be used to extract tissue for examination to determine the presence or extent of a disease, for example, cancer, cirrhosis, lymphoma, nephropathy and the like.

FIGS. 1A, 1B, and 10 depict the external components of a reusable biopsy device 100 in accordance with one embodiment. In contrast, FIG. 1D depicts an alternative implementation of a device 150, as discussed in additional detail below. In the depicted example of FIGS. 1A-10, the biopsy device 100 has a main body (or “housing”) 101 which is made up of a top half body portion (or “shell”) 102 and a bottom half body portion (or “shell”) 104 that are coupled together to form two chambers 105, 109 within the body 101: a first or distal chamber 105 and a second or proximal chamber 109 as shown. Alternatively, the housing 101 can be a single unitary component. In the illustrated example in FIGS. 1A-10, the device 100 also has a biopsy needle 107 extending through an opening 114 in the distal portion of the body 101. The needle 107 is made up of an outer cannula 110 with an inner needle 108 slidably disposed within the cannula 110. Further, the device 100 has a handle (or “plunger”) 112 that is moveable among three positions: a first or “extended” position as shown in FIG. 1A, a second or “retracted” position as shown in FIG. 1B, and a third (“tissue collection” or “intermediate”) position as shown in FIG. 10. As will be discussed in further detail below, the handle 112 is coupled to the inner needle 108 such that the inner needle 108 can be urged into an extended position (as shown in FIG. 1A) a retracted position (as shown in FIG. 1B), or an intermediate (or “tissue collection”) position (as shown in FIG. 10) when the handle 112 is moved among its three corresponding positions. As such, the inner needle 108 can be slidably moved along the outer cannula 110 into any of the three positions. The three positions of the handle 112 and the inner needle 108 will be discussed in further detail below. According to certain exemplary implementations such as the one depicted here, the main body 101 has one or more notches or indentations 111 defined along each side that are configured to assist a user in holding or gripping the device 100.

As shown in FIGS. 1A-1C, the three positions of the handle 112 result in three corresponding positions of the biopsy needle 107 for use in collecting a tissue sample. More specifically, when the handle 112 is in the extended position, the needle 107 (including both the inner needle 108 and the cannula 110) is in the extended position as shown in FIG. 1A. Further, when the handle 112 is in the retracted position, the needle 107 (including both the inner needle 108 and the cannula 110) is in the retracted position as shown in FIG. 1B such that the length of the needle 107 extending from the distal end of the body 101 in the retracted position is less than the length of the needle 107 in the extended position. Further, as can be seen in both FIGS. 1A and 1B, the inner needle 108 extends only a short length out of the cannula 110 in both the extended and retracted positions. However, when the handle 112 is in the intermediate position, which is the tissue sample collection position, the inner needle 108 extends out of the cannula 110 as shown in FIG. 10 such that a needle channel (also referred to herein as a “tissue collection channel”) 116 of the inner needle 108 extends out of the cannula 110. A close-up view of the needle 107 in the tissue sample collection position is depicted in FIG. 18B with the inner needle 108 extending from the cannula 110 such that the tissue collection channel 116 is disposed in the deployed position for collection of tissue.

In one implementation, FIGS. 2A-9 depict the various inner components and mechanisms of the device 100, including the energy storage and firing mechanism 120, as best shown in FIGS. 4, 6, 8, and 9. The energy storage and firing mechanism 120 is made up of two elongate tensionable members 106A, 106B (a distal tensionable member 106A disposed in the distal chamber 105 and a proximal tensionable member 106B disposed in the proximal chamber 109) and a cannula carrier (also referred to as a “sled,” “piston,” or “cannula piston”) 124, which will be described in additional detail below.

FIGS. 2A, 2B, and 2C depict the inner components of the device 100 in the three different positions, with FIG. 2A depicting the extended position, FIG. 2B depicting the retracted position, and FIG. 2C depicting the intermediate (tissue collection) position. When the handle 112 and needle 108 are in the extended position of FIG. 2A, the energy storage and firing mechanism 120 is in its deployed or distal position such that each of the elongate tensionable structures 106A, 106B is disposed at or near the distal walls of its respective chamber 105, 109, the cannula piston 124 is disposed against the distal inner wall 128A of the handle cavity (also referred to as a “piston cavity”) 128 (as discussed in further detail below), and the cannula 110 is in its extended position. Further, when the handle 112 and inner needle 108 are in the retracted position of FIG. 2B, the energy storage and firing mechanism 120 is in its retracted or proximal position such that each of the elongate tensionable structures 106A, 106B is disposed at or near the proximal walls of its respective chamber 105, 109, the cannula piston 124 is disposed against the distal inner wall 128A of the piston cavity 128 (as discussed in further detail below), and the cannula 110 is in its retracted position. In addition, when the handle 112 and needle 108 are in the intermediate position of FIG. 2C, the energy storage and firing mechanism 120 remains in its retracted or proximal position such that each of the elongate tensionable structures 106A, 106B is disposed at or near the proximal walls of its respective chamber 105, 109, the cannula piston 124 is disposed against the proximal inner wall 128B of the piston cavity 128 (as discussed in further detail below) and the cannula 110 is in its retracted position, while the needle 108 is in its extended position, as will be discussed in additional detail below.

FIG. 3, in according to certain embodiments, depicts an exploded view of the inner components and mechanisms of the device 100. The bottom half 104 of the body 101 has a channel 130 defined therein that is configured to slidably receive the handle 112 such that the handle 112 can move along the channel 130 from its extended position to its retracted position (and the intermediate position therebetween). As discussed above, the handle 112 has an opening or cavity 128 defined therein that is configured to receive the cannula piston 124 such that the piston 124 is slidably disposed within the cavity 128, as best shown in FIG. 4.

Also shown is the biopsy needle 107 (which is also similarly depicted in FIG. 7), with the cannula 110 and the inner needle 108 slidably disposed within the cannula 110 such that the distal tip of the needle 108 extends from the distal end of the cannula 110 and the proximal end of the needle 108 extending proximally from the proximal end of the cannula 110. The cannula 110 has a proximal cannula attachment body 113 at its proximal end and the inner needle 108 has a proximal needle attachment body 103 at its proximal end.

The cannula carrier 124 has an opening (also referred to herein as a “cavity” or “cannula cavity”) 126 defined therein that is sized and shaped to receive the proximal cannula attachment body 113 such that the body 113 can be disposed within the opening 126 of the carrier 124 (as best shown in FIGS. 4-6). Thus, the positioning of the body 113 within the carrier 124 results in the cannula 110 being attached to the cannula carrier 124. In addition, the carrier 124 has two slots 131A, 131B defined within the carrier 124 that are configured to receive the elongate tensionable members 106A, 106B such that the elongate tensionable members 106A, 106B are attached to the carrier 124 via the slots 131A, 131B (as best shown in FIGS. 4-6 and 8).

Further, the handle 112 has an opening (also referred to herein as a “cavity” or “needle cavity”) 127 defined therein as shown that is sized and shaped to receive the proximal needle attachment body 103 such that the body 103 can be disposed within the opening 126 (as best shown in FIG. 4). Thus, the positioning of the body 103 within the opening 127 of the handle 112 results in the needle 108 being attached to the handle 112.

The slidable relationship between the cannula carrier 124 and the handle 112 creates the slidable relationship between the cannula 110 and the inner needle 108. That is, as mentioned above, the cannula 110 is fixedly attached to the cannula carrier 124 (via the cannula body 113), while the inner needle 108 is fixedly attached to the handle 112 (via the needle body 103). And the cannula carrier 124 is slidably or movably disposed within the handle cavity 128 such that the carrier 124 is moveable in relation to the handle 112 and, as a result, the cannula 110 is slidable in relation to the inner needle 108.

When the handle 112 is moved, it can urge the cannula carrier 124 either distally or proximally once either the proximal or distal inner walls 128A, 128B of the handle cavity 128 make contact with the carrier 124. For example, when the handle 112 and inner needle 108 are in the extended position of FIG. 2A, the cannula carrier 124 is disposed against the distal inner wall 128A of the handle cavity 128. Thus, when the handle 112 is urged proximally into its retracted position of FIG. 2B, the distal inner wall 128A is in contact with and urges the cannula carrier 124 proximally such that the handle 112 ends up in the retracted position while the carrier 124 remains in contact with the distal inner wall 128A as shown in FIG. 2B. This proximal movement of the carrier 124 urges the tensionable members 106A, 106B proximally until they are disposed at or near the proximal walls of their respective chambers 105, 109 as shown.

In contrast, when the handle 112 is urged distally into its intermediate (tissue collection) position of FIG. 2C, the carrier 124 remains stationary such that the proximal inner wall 128B of the handle cavity 128 is urged into contact with the carrier 124 (while the tensionable members 106A, 106B remain in their proximal positions as shown). As discussed in further detail below, this results in the inner needle 108 extending out of the cannula 110 such that the tissue collection channel 116 is exposed.

The cannula carrier 124 (and the tensionable members 106A, 106B) move freely in relation to the handle 112 because the side walls of the carrier 124 remain spaced from and are not in contact with any portion of the handle 112 (or any other part of the device 100) during movement except at the ends of the carrier 124, and further because the tensionable members 106A, 106B also remain spaced from and are not in contact with the upper or lower surface of the body 101 (or any other part of the device 100) during movement. Put another way, both the carrier 124 and the tensionable members 106A, 106B are “floating” inside the device body 101 such that they are not hindered by contact friction with any other surfaces of the device 100. This eliminates frictional losses during firing of the device 100, while other known devices typically have to overcome the friction created by the various moving parts therein.

As noted above, the tensionable members 106A, 106B are tensionable structures (also referred to herein as “flexures” or “flexure components”) that can be moved between two positions: a distal position when the handle 112 is in the extended position as shown in FIG. 2A, and a proximal position when the handle 112 is in either the retracted position as shown in FIG. 2B or the tissue collection position as shown in FIG. 2C. The flexure components 106A, 106B are untensioned or “stable” in both the extended and retracted positions, and thus are “bistable” components with two untensioned or stable positions. In contrast, in any position between the distal and proximal positions, the two flexure components 106A, 106B are tensioned such that they are urged toward their distal positions. Thus, when a user pulls the handle 112 back into the retracted position, the flexure components 106A, 106B are urged from one stable configuration (the distal position) to the other (the proximal position), where the handle 112 and flexure components 106A, 106B rest in the retracted or “cocked” position. At this point, when the device 100 has been positioned as desired, the user can urge the handle 112 distally such that it is urged into its tensioned position and thus the flexure components 106A, 106B urge the handle 112, the cannula carrier 124, and thus the cannula 110 distally until the flexure components 106A, 106B arrive back at their distal positions. According to certain embodiments, the flexure components 106A, 106B are sufficiently tensioned such that they urge the cannula 110 distally with greater force and velocity than most standard biopsy devices, thereby causing the cannula 110 to enter the target tissue with a powerful cutting motion that results in the severing of a tissue sample.

Another feature of the current device 100 that increases the force and velocity of the flexure components 106A, 106B (and thus the cannula 110) is the distance that the flexure components 106A, 106B are allowed to travel. More specifically, the distance between the proximal walls and the distal walls of the two body chambers 105, 109 are configured to allow the flexure components 106A, 106B to travel the full distance therebetween, which allows the flexure components 106A, 106B to attain a greater velocity and force in comparison to a shorter distance. In accordance with one specific embodiment, the flexure components 106A, 106B travel a distance of about 20 mm from their proximal positions to their distal positions.

In certain implementations, the flexure components 106A, 106B are made of a tensionable, flexible metal. In one specific example, the flexure components 106A, 106B are made of stainless steel, and in certain more specific embodiments, of 301 stainless steel. Alternatively, the flexure components 106A, 106B can be made of 304 stainless steel, 430 stainless steel, or a titanium alloy. In a further alternative, the flexure components 106A, 106B can be made of any tensionable, flexible material having a high elastic modulus similar to 301 stainless steel.

The velocity and force created by the flexure components 106A, 106B can also be modified or adjusted by stacking more than one flexure component 106A, 106B together. More specifically, as shown in one exemplary embodiment in FIG. 9, the distal flexure component 106A can actually be made up of two flexure components 106A that are coupled or “stacked” together, and the proximal flexure component 106B can be also be made up of two flexure components 106B coupled together. Alternatively, one or both of the flexure components 106A, 106B can be made up of three or more flexure components. In a further alternative, only one of the two flexure components 106A, 106B can be made up of more than one flexure components. The use of two flexure components instead of one results in an increase in force generated by the stacked flexure component that is substantially twice the amount of force generated by a single, unstacked flexure component. Similarly, the use of three flexure components stacked together results in about triple the force. According to one embodiment, the number of flexure components can range from 1 to 8 components stacked together. Alternatively, any number of flexure components that can be disposed within any device embodiment herein can be stacked together.

Given the minimal thickness of each flexure component, this stacking feature makes it possible to substantially increase the force generated by the flexure components without requiring any increase in the overall size of the device 100. According to certain embodiments, each flexure component has a thickness ranging from about 0.0005 inches to about 0.015 inches. Alternatively, each flexure component has a thickness ranging from about 0.004 inches to about 0.010 inches. In a further alternative, the thickness can range from about 0.006 to about 0.008 inches. In a further alternative, the thickness of the flexure component is about 0.007 inches. Thus, using the 0.007 inch thickness as an example, doubling the force by stacking two flexure components together requires only 0.014 inches of flexure thickness, and tripling the force (by stacking three) requires only 0.021 inches, both of which are relatively negligible increases in space consumed within the device 100. In contrast, known, commercially-available biopsy devices typically rely on compression springs to generate cutting force, and doubling the output force of such a spring requires far greater space requirements.

According to certain embodiments, each flexure component can have an actual length (or “cut length”) of about 74 mm to about 110 mm. Alternatively, each component can have an actual length of about 80 mm to about 100 mm. Further, in certain implementations, each flexure component when disposed within any device embodiment herein can have a free length (the length of the flexure that is not attached to or in contact with any portion of the device) ranging from about 45 mm to about 65 mm. Alternatively, each component can have a free length of about 50 mm to about 60 mm. In addition, according to certain embodiments, each flexure component can have a width ranging from about 2 mm to about 15 mm. Alternatively, each component can have a width of about 5 mm to about 12 mm. In accordance to various implementations, the attachment angle of the flexure components at the ends thereof can range from about −60 degrees to about 60 degrees. Alternatively, the angle can range from about −40 degrees to about 40 degrees.

FIGS. 10A and 10B depict cross-sectional views of the device 100, with FIG. 10A depicting the longitudinal cross-section along line A as shown in FIG. 1A and FIG. 10B depicting the radial cross-section along line B in FIG. 1A.

In use, the device 100 can be used to collect a tissue sample in the following manner. Prior to use, the device 100 is typically disposed in its initial or “extended” position as shown in FIGS. 1A and 2A. In this position, as best shown in FIG. 2A, the cannula carrier 124 is disposed at its distal position within the cavity 128 of the handle 112 such that the distal end of the carrier 124 is in contact with the distal inner wall 128A of the cavity 128. More specifically, the flexure components 106A, 106B are in their distal position such that the components 106A, 106B retain the carrier 124 in contact with the distal inner wall 128A and are tensioned as described elsewhere herein so as to retain the handle 112 in this extended position unless and until sufficient force is applied to urge the handle 112 by a user.

When the user is ready to take a tissue sample from a patient, the user first retracts the handle 112 into its retracted position. That is, the user grabs the handle 112 and urges it proximally from its initial or extended position (as shown in FIGS. 1A and 2A) into its retracted position as shown in FIGS. 1B and 2B. This proximal movement of the handle 112 causes the proximal movement of the cannula carrier 124, because the distal inner wall 128A is in contact with the carrier 124 and thus the proximal movement of the wall 128A urges the carrier 124 proximally as well. Because of the flexure components 106A, 106B, the force required to be applied by the user in order to retract the handle 112 (and cannula carrier 124) grows larger as it is displaced, until the flexures 106A, 106B “snap” or pass through their singular configuration, bringing the flexures 106A, 106B (and the cannula carrier 124) into the cocked bistable position in which the flexures 106A, 106B are disposed in the proximal position adjacent to the proximal walls of each of the chambers 105, 109 (as shown in FIG. 2B). Retracting the handle 112 in this fashion causes the retraction of the inner needle 108 and the cannula 110 together, causing no relative displacement between them.

It should be noted at this point that when the handle 112 (and carrier 124) are in this retracted position, the handle 112 can be freely moved (with minimal force) distally with respect to the carrier 124 between the retracted position (as shown in FIGS. 1B and 2B) and the intermediate or tissue collection position as shown in FIGS. 10 and 2C. That is, as best shown in FIG. 2C, while the carrier 124 is retained in its proximal position by the flexure components 106A, 106B, the handle 112, as a result of the space provided in the handle cavity 128 for the carrier 124, can move freely between its retracted position (of FIG. 2B) and its intermediate position (of FIG. 2C). This freedom of movement of the handle 112 in relation to the carrier 124 thereby allows the movement of the inner needle 108 in relation to the cannula 110.

Once the physician (or other user) has retracted the handle 112 (and carrier 124, inner needle 108, and cannula 110) into the retracted position, the physician then pierces the skin of the patient with the tip of the needle 107 (the inner needle 108 and cannula 110) and urges the tip of the needle 107 distally into the patient until the tip is positioned at or near the edge of a target tissue (such as, for example, a solid tumor). Once the tip of the needle 107 is positioned as desired, the physician holds the device 100 in place with respect to the patient while urging the handle 112 distally to the tissue collection position depicted in FIGS. 10 and 2C in which the proximal inner wall 128B of the handle cavity 128 makes contact with the proximal end of the carrier 124. This movement of the handle 112 causes the inner needle 108 to extend distally out of the cannula 110 and thus extend into the target tissue (such as a tumor) while exposing the tissue collection channel 116 in that target tissue.

Once the inner needle 108 has been inserted into the target tissue such that the tissue collection channel 116 extends out of the cannula 110 and into the target tissue, the physician can “fire” the device 110 to collect tissue within the channel 116. That is, the physician can urge the handle 112 distally such that the handle 112 urges the cannula carrier 124 distally as well (since the proximal inner wall 128B is already in contact with the proximal end of the carrier 124). This distal movement of the handle 112 and carrier 124 urges the flexures 106A, 106B distally out of their stable proximal position, thereby urging the flexures 106A, 106B into a tensioned state in which they are quickly urged distally (or quickly “snap” forward) as a result of that tensioned state such that they return to their untensioned distal position (as shown in FIGS. 1A and 2A). This movement of the flexures 106A, 106B is extremely forceful and fast as a result of the tensioned nature of the components 106A, 106B as they are urged distally from their proximal positions, thereby urging the cannula 110 distally with sufficient force to create the necessary cutting power by the cannula 110 to sever tissue disposed within the tissue collection channel 116, entrapping the tissue in the cavity within the cannula 110 created by the channel 116.

Once the device 100 returns to the extended position such that the collected tissue is entrapped within the cannula 110, the physician (or other user) removes the device 100 from the patient by urging the device proximally in relation to the patient, thereby retracting the needle 107 (both the cannula 110 and inner needle 108) from the patient's tissue. Once removed, the handle 112 can be urged proximally back into its retracted position such that the cannula 110 is retracted in relation to the inner needle 108, thereby exposing the severed tissue disposed in the channel 116. At this point, the severed tissue can be removed from the channel 116 and transported to the appropriate location for testing.

An alternative biopsy device 150 embodiment is depicted in FIG. 1D. This device 150 is substantially similar to the device 100 discussed above with respect to its components, features, and functionality, except as expressly discussed herein. In this specific implementation as shown, the top shell 152 has a slot 154 defined within the shell 152. More specifically, the slot 154 extends along a length of the shell 152 and is sized and shaped such that the various components of the biopsy needle 157 can pass therethrough. That is, the slot 154 is shaped as shown in FIG. 1D such that the cannula 160 with the proximal cannula attachment body 163 and the inner needle 158 with the proximal needle attachment body 153 can pass through the slot 154. As such, during use of the device 150, a biopsy needle 157 can be inserted through the slot 154 to be attached to the device 150 for use, and also can be removed from the device 150 through the slot 154 for replacement, repair, and/or maintenance of the needle 157 and/or the device 150, all without having to remove the top shell 152 or otherwise dismantle the body 151.

In certain implementations, the device 150 also has a flexible attachment protrusion (or “clip”) 156 flexibly disposed at the proximal end of the device 150. More specifically, the protrusion 156 extends distally into a proximal portion of the slot 154 and thus is disposed over a portion of the proximal needle attachment body 153 when the needle 157 is positioned within the device 150. In other words, the flexible attachment clip 156 helps to retain the needle 157 within the device 150. Further, the flexibility of the clip 156 allows for a user to urge the clip 156 upwards and away from the slot 154 to thereby clear the space above the needle attachment body 153 and allow for the needle 157 to be inserted into or removed from the device 150 through the slot 154.

A further alternative version of the device is depicted in FIGS. 11A and 11B, in which the device 100 is substantially similar to the device 100 of FIGS. 1A-10B except for the ratcheting components 125. That is, in this specific alternative embodiment, the proximal end of the cannula carrier 124 has two flexible retention projections 125 as shown. As best shown in FIG. 11A, the projections 125 are disposed in a untensioned state in which the total width of the projections 125 is greater than the width of the sides of the carrier 124 and further is greater than the width of the proximal opening 123 (as best shown in FIG. 3) defined in the body 101 of the device 100. Further, the projections 125 are flexible such that they can be urged inward (toward each other) by an external force, thereby reducing their total width, but are tensioned such that they will return to their untensioned state (as shown in FIGS. 11A and 11B) upon removal of that force. As a result, when the handle 112 and carrier 124 are urged proximally into the retracted position (as shown in FIGS. 11A and 11B), the projections 125 make contact with the walls of the proximal opening 123 and are urged inwardly until the projections 125 pass through the opening 123, at which point the projections 125 expand back to their expanded, untensioned state. At this point, the projections 125 have a width greater than the proximal opening 123, thereby causing the carrier 124 to be retained in that position (with the projections 125 disposed externally of the body 101 as shown). The retention of the carrier 124 (and thus the handle 112) in this position can help to prevent an accidental actuation (or “misfire”) of the device 100. Alternatively, any known retention mechanisms or structures can be used in place of these projections 125. Regardless, the retention projections 125 (or other similar mechanism) are a safety feature that prevent an inadvertent actuation of the device 100. According to one embodiment, to disengage the projections 125 and actuate the device 100, the handle 112 is urged distally, and two wedge structures 129 disposed on the handle 112 as shown make contact with the projections 125. When the wedge structures 129 contact the projections 125, they urge the projections 125 inwardly (toward each other), thereby reducing their overall width until they can pass through the proximal opening 123. And further urging the handle 112 distally urges the carrier 124 a sufficient distance to cause the flexures 106A, 106B to actuate the carrier 124 and handle 112 to “snap” forward or “fire.”

Another embodiment of a reusable biopsy device 200 is depicted in FIGS. 12A-17. In this exemplary implementation, the biopsy device 200 has a main body housing 201 made up of a top half body shell 202 and a bottom half body shell 204. Alternatively, the biopsy device 200 may be comprised of a singular body shell 201, such that the main body 201 is integrally formed of a singular piece of material. In the illustrated example, the main body 201 houses one or more flexure components 206 and a needle 207 that is made up of an inner needle 208 and an outer cannula 210.

Any of the various components, features, and functionality described above with respect to the various device embodiments disclosed or contemplated above and/or depicted in FIGS. 1A-11B can also be incorporated into any of the embodiments disclosed or contemplated below and/or depicted in FIGS. 12A-18B. Similarly, any of the various components, features, and functionality described below with respect to the various device embodiments disclosed or contemplated below and/or depicted in FIGS. 12A-18B can also be incorporated into any of the embodiments disclosed or contemplated above and/or depicted in FIGS. 1A-11B.

FIG. 12A shows a perspective view of the reusable biopsy device 200 with a handle 212 in a first or extended position in accordance with one embodiment. In this exemplary implementation, the main body 201 is formed with one or more finger or hand notches or indentations 211 defined within the body 201 that are configured to assist a user in holding or gripping the device 200. In the depicted example, the inner needle 208 and outer cannula 210 extend from the main body 201 through an opening 214. In some embodiments, the outer cannula 210 is operably coupled to the handle 212 such that movement of the handle 212 causes corresponding movement of the cannula 210. In certain embodiments, the outer cannula 210 is movable in relation to the inner needle 208. The handle 212 may be configured to move between the extended position and a second or retracted position as depicted in FIG. 12B

FIG. 12B depicts the reusable biopsy device 200 with the handle 212 in the second or retracted position in accordance with one embodiment. More specifically, the handle 212 is extended or pulled away from the main body 201 to engage or tension the energy storage and firing mechanism 220, as will be described in additional detail below. In the depicted example, when the handle 212 is retracted into the position as shown in FIG. 12B, the outer cannula 210 is urged proximal (or retracted) in relation to the inner needle 208 such that the tissue sampling channel 216 of the inner needle 208 becomes exposed.

FIG. 13 shows a perspective view of the underside of the reusable biopsy device 200 with a trigger or actuator release 218 in a first position in accordance with one embodiment In some examples, the trigger 218 may be configured as a linkage or button and may be used to lock the cannula carrier 224 (as shown in FIG. 14) in a second position as described in additional detail below, wherein the cannula carrier 224 is retracted and thereby retracts the outer cannula 210 to expose the tissue collection channel 216 in the inner needle 108. In some examples, the trigger 218 may lock the cannula carrier 224 into a selected position of a set of predetermined positions. For example, the cannula carrier 224 can have protruding ratcheting elements 225 as shown in FIG. 14 that are configured to engage with the trigger 218 after a fixed displacement.

In some embodiments, the handle 212 may be configured to actuate the device 200. For example, the handle 212 may be advanced forward a first predetermined distance to advance the inner needle 208 forward. The handle 212 may then be advanced forward a second predetermined distance to actuate the automatic release of the outer cannula 210.

FIG. 14 shows an exploded view of the reusable biopsy device 200 in accordance with one embodiment in which the inner needle 208 has a proximal needle body 203 and the outer cannula 210 has a proximal cannula body 213. In addition, the device 200 has an energy storage and firing mechanism 220 having one or more flexure components 206, a cannula carrier 224, and one or more cannula attachment points 226. In the depicted example, the energy storage and firing mechanism 220 has two flexure components 206 and two cannula attachment points 226. In some examples, the flexure components 206 may be formed in flexed, curved or serpentine orientations. Alternatively, the flexure components 206 can have any configuration that allows for the flexure components 206 to be tensioned as described herein. That is, in any embodiment of the device 200, the flexure components 206 may be adapted to bias the cannula 210 towards its extended position, wherein the cannula 210 covers the shelf or channel 216 of the inner needle 208. In the depicted example, the flexure components 206 include a groove 222 defined within each of the flexure components 206. In some embodiments, the groove 222 enables or enhances the flexibility of the flexure components 206. In the depicted example, the cannula carrier 224 includes the cannula attachment points 226 extending from one side of the carrier 224 as shown and one or more protruding ratchet elements 225 extending from the opposing side of the carrier 224. In some embodiments, the protruding ratchet elements 225 are disposed along the cannula carrier 224 in pairs.

In accordance with any device embodiments herein, the energy storage and firing mechanism comprises a biasing module. In certain embodiments, the biasing module comprises one or more compliant flexure components as discussed above in relation to device 100 and, in another embodiment, device 200. In some embodiments, the biasing module comprises a pair of flexure components. In some embodiments, the biasing component may further comprise one or more additional tension components, for example, one or more tension or compression springs.

The flexure components of the various implementations herein may be configured both for potential energy storage and to direct the motion or movement of the biopsy device's inner needle or outer cannula. In some embodiments, the range of motion of the cannula and firing force directly correlate to the nonlinear stiffness of the flexure components. In some scenarios, the stiffness of the flexure components depends on the thickness of the components, as well as their length, curvature, cross-sectional geometry, material, and loading direction. One of ordinary skill in the art would appreciate that there are numerous measurements, configurations or materials that might be used to form the flexure components, which may depend on the intended application of the biopsy device, and embodiments thereof are contemplated for use with any such measurements, configuration, or materials.

As further shown in FIG. 14, the bottom half body shell 204 may include one or more needle attachment points 227, a trigger slot 228 defined within the shell 204, and a cannula carrier path 230 defined in or disposed on the shell 204. The trigger slot 228 may be configured to receive or otherwise provide access to the trigger or actuator release 218. In the depicted example, the bottom half body shell 204 has two needle attachment points 227. In some embodiments, the cannula attachment points 226 discussed above are protrusions configured to connect to corresponding holes in the cannula connector 213 of the outer cannula 210. Similarly, in some embodiments, the needle attachment points 227 are protrusions configured to connect to corresponding holes in the needle connector 203 of the inner needle 208, respectively. In any embodiment, any other known, suitable attachment members may be used to connect the needle 208 to the bottom half body shell 204 and the outer cannula 210 to the cannula carrier 224. In accordance with various embodiments, the cannula carrier 224 may be configured to move along the cannula carrier path 230 to selectively extend and retract the cannula carrier 224 connected to the outer cannula 210, as desired by a user.

FIGS. 15A and 15B show a perspective view of the reusable biopsy device 200 with the top half body shell removed to show the interior components of the main body housing 201. In FIG. 15A, the device 200 is depicted in the first or extended position and in FIG. 15B, the device 200 is depicted in the second or retracted position. Turning first to the device in the extended position of FIG. 15A, the shelf or channel 216 of the inner needle 208 is covered by the outer cannula 210 in accordance with one embodiment. In the depicted example, the needle connector 203 of the inner needle 208 is connected to the bottom half body shell 204 at the needle attachment points 227 and the cannula connector 213 of the outer cannula 210 is connected to the cannula carrier 224 at the cannula attachment points 226. In the depicted example, the handle 212, the energy storage and firing mechanism 220, the cannula carrier 224 and the cannula 210 are in the first or extended position, wherein the handle 212 is in its extended or distal position, the flexure components 206 are in their untensioned position, the cannula carrier 224 is positioned towards a distal portion of the device 200, and the cannula 210 covers the shelf or channel 216 of the needle 208. As shown in FIG. 15A, in the first position, the flexure components 206 may bias the outer cannula 210 distally, for example, by biasing the cannula carrier 224 towards a distal portion of the device 200 such that the outer cannula 210 covers the shelf 216 of the inner needle 208.

In FIG. 15B, in which the device 200 is in its second or retracted position, the shelf or channel 216 of the inner needle 208 is exposed in accordance with one embodiment. In the depicted example, the handle 212, the energy storage and firing mechanism 220, the cannula carrier 224, and the cannula 210 are in a second or retracted position, wherein the handle 212 is in its retracted or proximal position, the flexure components 206 are engaged and in their expanded or tensioned position, the cannula carrier 224 is positioned towards a proximal portion of the device 200, and the cannula 210 is moved to expose the shelf or channel 216 of the needle 208. In this position, the flexure components 206 are tensioned such that they are configured to bias the cannula carrier 224 distally, such that when the flexure components 206 are released, a cannula firing force is discharged and the cannula carrier 224 directs the cannula 210 forward (distally) such that the sharp edge of the cannula 210 advances quickly, for example, to sever the cylindrical “core” of tissue that has been placed in the recessed shelf 216 of the inner needle 208.

In accordance with any embodiments disclosed or contemplated herein, the biopsy needle (including the cannula and the inner needle) may be guided into subject tissue by the use of an introducer needle. In some embodiments, the introducer needle may comprise a hollow cannula and a removable inner sharp stylet. The introducer needle may be utilized to place the biopsy needle into the target tissue, and may be fully removed to permit the introduction of the biopsy needle. The introducer needle may have a plastic hub at its proximal end, that the handle or main body housing of the biopsy device may seat itself into during placement of the biopsy needle into the target tissue, in some scenarios, necessitating the need for a precisely matched introducer length for the biopsy needle. The introducer needle may be wider or larger than the biopsy needle and configured to house the biopsy needle. For example, the introducer needle may be one gauge larger than the biopsy needle to adequately house the biopsy needle.

FIG. 16 shows a partially exploded view from above the reusable biopsy device 200 in accordance with an embodiment. In the depicted example, the bottom half body shell 204, the energy storage and firing mechanism 220, the handle 212, the needle 208, the cannula carrier 224, and the cannula 210 are shown.

FIG. 17 shows a partially exploded view from the underside of the reusable biopsy device 200 in accordance with one embodiment. In the depicted example, the bottom half body shell 204 includes a trigger or actuator release 218 configured to trigger the hold or release of the energy storage and firing mechanism 220. For example, the trigger 218 may be configured to reversibly lock the cannula carrier 224 and the cannula 210 in place in a predetermined position based on the location of the trigger 218 with respect to the protruding ratchet elements 225 on the cannula carrier 224. Moreover, the trigger 218 may be used to release the energy storage and firing mechanism 220 such that the cannula carrier 224 is released to direct the outer cannula 210 forward to enable the sharp edge of the outer cannula 210 to advance quickly to sever the cylindrical “core” of tissue that has been placed in the shelf 216 of the inner needle 208.

As mentioned above, FIGS. 18A and 18B depict a perspective view of a biopsy needle 207 of a reusable biopsy device (such as device 100 or device 200, for example) in two different configurations in accordance with one embodiment. As shown in FIG. 18A, the biopsy needle 207 has an inner needle 208, for example, an obturator or stylet, and an outer cutting cannula 210. In some embodiments, the biopsy needle 207 may be disposable. In the depicted example, the cannula 210 is in the extended position such that the shelf or channel 216 of the inner needle 208 is covered by the outer cannula 210.

In contrast, FIG. 18B shows a perspective view of the biopsy needle 107 in the collection position in which the cannula 210 is retracted such that the channel 216 of the inner needle 208 is exposed. That is, the inner needle 208, and any inner needle embodiment herein, has a notch, shelf or channel 216 defined near the distal end of the needle 208 that is configured to fill with tissue when the channel 216 is exposed and is advanced into a biopsy site. In the illustrated example, the outer cannula 210 is sharp at one of its ends or edges. In some embodiments, the outer cannula 210 may be manually retracted back from the inner needle 208 by a set distance. In some scenarios, engaging the energy storage and firing mechanism 220, for example, and then triggering or releasing the expanded flexure components 206 of the device 200 directs the sharp edge of the cannula 210 to advance in the distal direction quickly, for example, to sever the cylindrical “core” of tissue that has been placed in the recessed shelf 216 of the inner needle 208.

In accordance with an exemplary usage scenario, a user may load or tension the energy storage mechanism 220 (the flexure components 206) by urging the handle or plunger 212 into its retracted position. Once the energy storage mechanism 220 has accumulated enough potential energy (has been sufficiently tensioned), through the flexure components 206 (or other tensionable components) undergoing some displacement, the handle 212 can be locked or otherwise maintained in place in that retracted position by, for example, the ratcheting protrusions 225 that are configured to engage with the handle 212 or the trigger 218. In some examples, the retraction of the handle 212 into the retracted position can be a displacement of 10, 15, or 20 millimeters from the extended position, and may depend on the size of biopsy sample being collected. That is, the amount of retraction can, in certain embodiments, be varied as desired. In some examples, the outer cannula 210 may be operably connected or rigidly fixed to the displaced end of the energy storage and firing mechanism 220, for example, at a designated position on the cannula carrier 224. Thus, retracting the handle 212 to load the energy storage and firing mechanism 220 can also retract the cannula 210. In some examples, the needle 208 may be free to be manually positioned within the cannula 210.

Further in accordance with the exemplary usage scenario, the inner needle 208 and the outer cannula 210 may be positioned such that the tip of the needle 208 is approximately aligned with the tip of the cannula 210, and the cannula 210 and needle 208 may be inserted into the biopsy site to a desired depth, for example, towards the edge of a tumor or biopsy site. In some examples, the outer cannula 210 and inner needle 208 may be manually advanced to the desired depth. In some scenarios, the shelf or channel 216 of the needle 108 may then be exposed to the biopsy tissue by retracting (engaging) the handle or plunger 212 to manually move inner needle 208 (or move the cannula 210 in relation to the needle 208) to expose the shelf or channel 216 of the inner needle 208. Because tissue is compliant, it may expand to fill the shelf or channel 216 of the inner needle 208.

Further in the exemplary usage scenario according to one embodiment, a user may release the retracted handle 212 or the trigger 218 holding the energy storage and firing mechanism 220 in its high energy or tensioned state, to release the potential energy of the flexure components 206 of the energy storage and firing mechanism 220 in the form of a rapid forward motion, which may drive the outer cannula 210 forward, since the outer cannula 210 is connected to the cannula carrier 224 of the energy storage and firing mechanism 220. In some implementations, the use of the trigger 218 to release the ratcheting protrusions 225 causes the outer cannula 210 to shoot forward along the inner needle 208, to rapidly sever the tissue sample and contain the sample within the shelf or channel 216 of the inner needle 208. The biopsy device 200 may then be removed from the site and the shelf or channel 216 of the inner needle may be exposed again, this time for the user of the device 200 to collect the tissue sample.

According to an embodiment of the invention, one or more of the main body housing 201, the flexure components 206, the handle 212, or any parts thereof, may be formed from a suitable thermoplastic material, which may include, for example, Acrylanitrile Butadiene Styrene (ABS), Polycarbonate (PC), Mix of ABS and PC, Acetal (POM), Acetate, Acrylic (PMMA), Liquid Crystal Polymer (LCP), Mylar, Polyamid-Nylon, Polyamid-Nylon 6, Polyamid-Nylon 11, Polybutylene Terephthalate (PBT), Polycarbonate (PC), Polyetherimide (PEI), Polyethylene (PE), Low Density PE (LDPE), High Density PE (HDPE), Ultra High Molecular Weight PE (UHMW PE), Polyethylene Terephthalate (PET), PolPolypropylene (PP), Polyphthalamide (PPA), Polyphenylenesulfide (PPS), Polystyrene (PS), High Impact Polystyrene (HIPS), Polysulfone (PSU), Polyurethane (PU), Polyvinyl Chloride (PVC), Chlorinated Polyvinyl chloride (CPVC), Polyvinylidenefluoride (PVDF), Styrene Acrylonitrile (SAN), Teflon TFE, Thermoplastic Elastomer (TPE), Thermoplastic Polyurethane (TPU), Engineered Thermoplastic Polyurethane (ETPU), Ethylene Chlorotrifluoroethylene (ECTFE), Ethylene tetrafluoroethylene (ETFE), Polychlorotrifluoroethylene (PCTFE), Fluorinated ethylene propylene (FEP), Polyether Ether Ketone (PEEK), Perfluoroalkoxy alkanes (PFA), Polyphenylene sulfide (PPS), Polyphenylsulfone (PPSU), Polysulfone (PSU), Polyetherimide (PEI), Ultem, polytetrafluoroethylene (PTFE), or any combination thereof. In any embodiment, the thermoplastic may be autoclavable or otherwise nondestructively sterilized for reuse by any means or methods, such as, for example, sterilization via ethylene oxide gas or ionizing radiation. Alternatively, the flexure components 206 can be made of any flexible metal in the same fashion as the flexure components 106 discussed above, including, for example, stainless steel.

In some embodiments, the main body housing in any embodiment herein can be formed of an autoclavable thermoplastic material. In any embodiment, the main body housing may be formed of any sterilizable material. One of ordinary skill in the art would appreciate that there are numerous configurations or materials that might be used to form the main body housing, and various embodiments are contemplated for use with any such configuration or materials.

According to various embodiments, any biopsy device embodiment or parts thereof disclosed herein may be designed with precise measurements, for example, thickness, stiffness, or length, based on the device's intended application. In some embodiments, portions of the biopsy device, for example, the energy storage and firing mechanism of the biopsy device may be designed using computer-aided design (“CAD”) techniques. In some embodiments, components of the biopsy device may be configured in a CAD model that can then be used to control 3D printing manufacturing process. Finally, the use of CAD models in conjunction with 3D printing, may allow for the precise reproduction of identical biopsy devices.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In accordance with any device embodiments disclosed or contemplated herein, the biopsy device (such as device 100, device 200, or any contemplated device) may be semi-automatic. For example, the inner needle of the device may be advanced forward manually, while the outer cutting cannula of the device may be advanced forward automatically through the use of the biasing force produced by the flexure components of the energy storage and firing mechanism.

In accordance with any device embodiments herein, the energy storage and firing mechanism may be configured as a compliant “living-hinge.” Further, the flexure components in any device implementation herein may be formed of a thin plastic material to allow the carriage to undergo large displacements, for example, displacements of approximately 20 millimeters. In further alternatives, the energy storage and firing mechanism may not depend on planar surfaces sliding against one another, such that the device may not be vulnerable to jamming or seizing. Thus, the various biopsy device embodiments may not require lubrication or regular maintenance prior to re-use. In further implementations, the device has a zero percent misfire rate.

In accordance with certain embodiments of the various devices disclosed or contemplated herein, the device may be lightweight. For example, in certain implementations, the device may weigh less than thirty grams. One of ordinary skill in the art would appreciate that there are numerous workable weights for the device, and embodiments thereof are contemplated having any such workable weight.

In accordance with certain embodiments of the various devices herein, the device may have a cannula firing speed anywhere between about 1.5 and about 20 meters per second. Alternatively, the speed may range from about 5 to about 17 meters per second. In a further alternative, the speed can range from about 10 to about 15 meters per second. One of ordinary skill in the art would appreciate that there are numerous workable cannula firing speeds for the device, and the embodiments herein are contemplated having any such workable cannula firing speeds.

In accordance with various implementations, the various device embodiments herein can have a biopsy needle with a cutting force of between about 15 and about 75 newtons. Alternatively, the cutting force can range from about 20 to about 60 newtons. In a further alternative, the cutting force can range from about 35 to about 50 newtons. One of ordinary skill in the art would appreciate that there are numerous cutting force potentials for the device, and the embodiments herein are contemplated for use with any such cutting force.

In accordance with various embodiments, the device (including device 100 or device 200) may be reusable. For example, the device may be configured to be reused between fifty to one hundred times. In some scenarios, the autoclavable thermoplastic that may be used to form portions of the device may allow the device to undergo the approximately fifty to one hundred uses and autoclave cycles before the end of the device's life cycle.

In accordance with various implementations, the device may comprise ten or less individual components. In some embodiments, the use of fewer components may minimize the complexity of the device's assembly, as well as reduce the manufacturing and purchase price per device unit. One of ordinary skill in the art would appreciate that there are numerous configurations or number of components that might be used to form the device, and the embodiments herein are contemplated for use with any such configuration or number of components.

In accordance with any of the embodiments herein, the various devices may be configured to be compatible with Magnetic Resonance Imaging (MRI), X-ray, Computed Tomography (CT), ultrasound, and nuclear medicine imaging, for example, Positron-Emission Tomography (PET) devices and/or machinery. One of ordinary skill in the art would appreciate that there are numerous configurations for the device embodiments that might make the device compatible with any other imaging devices or machinery, and the embodiments herein are contemplated for use with any such imaging devices or machinery.

### Example

A flexure testing jig was created to assess the displacement and force characteristics of various flexures for use in the various biopsy device embodiments herein. As shown in FIGS. 19A-19C, the flexure testing jig 300 is an adjustable frame 302 with four elongate members 304A, 304B, 306A, 306B, with each elongate member being an extruded aluminum t-slot member. The two opposing elongate members 304A, 304B are adjustable in relation to each other via movable attachment to the other two opposing elongate members 306A, 306B. Further, each of the two adjustable members 304A, 304B has two flexure attachment structures 308 attached thereto. Each of the structures 308 has multiple radial slots 310 defined with the structure 308 at fixed angles, wherein each slot 310 is configured to receive a flexure. As such, during testing, the flexures can be inserted into the desired slot 310 based on its angle, thereby allowing the flexures to be tested at multiple mounting angles. The distance between the two adjustable members 304A, 304B can be adjusted to test the flexures at varying attachment distances.

For testing purposes, the two flexures 312A, 312B are attached to each other via a carriage (or “sled”) 314 that can be used to move the two flexures 312A, 312B in a fashion similar to the movement of the flexures within certain biopsy device embodiments as disclosed or contemplated herein. As shown in FIG. 19A, both ends of both of the flexures 312A, 312B are attached to the attachment structures 308 at the same slot 310 and disposed in one of its bistable untensioned positions. In FIG. 19B, the sled 314 has been urged toward the elongate member 306B, thereby urging the flexures 312A, 312B into a tensioned position as shown. FIG. 19C depicts some of the measurements made relating to the various tests. For example, the measurements can include the distance between the two adjustable members 304A, 304B (also referred to as the “flexure width”) A, along with the angle at which each flexure is attached to the attachment structures 308 (also referred to as “flexure angle”) B. In addition, the distance of travel of the flexures 312A, 312B (also referred to as the “displacement distance” or “peak to peak displacement”) C. Other measurements include the force of the flexures 312A, 312B during travel, distance between the attachment structures 308 (also called the “free length”), the length of the flexures, the thickness of the flexures, the number of layered or stacked flexures, the flexure elastic modulus, and the flexure bistability. Another parameter that was tested was the flexure material (including various types of metals). More specifically, various flexible metals (with different moduli of elasticity, creep resistance, ductility, yield strength, etc) were tested to assess their suitability as use for flexures. All of the above parameters taken together comprise the “flexure design.” Identifying the optimal flexure designs that can apply adequate force over a suitable displacement distance required extensive testing using the jig and thorough data collection. Because computational simulation of flexures of this nature is very challenging (as a result of the “snap through” bistable behavior of the flexures being very difficult to model mathematically), flexure design experimentation is very helpful.

The testing results are set forth below in Table 1.

TABLE 1

| | | | Free | Actual | | Fixed | | | |
|---|---|---|---|---|---|---|---|---|---|
| | Thickness | | Length | Length | Width | Angle | Displacement | | |
| Test # | (in.) | Layers | (mm) | (mm) | (mm) | (°) | (mm) | Force | Bistable |
| 1 | .01 | 1 | 65 | 100 | 7 | 0 | 21.7 | Light | Y |
| 2 | .01 | 1 | 66.5 | 100 | 7 | 18 | 16.2 | Light | Y |
| 3 | .01 | 1 | 66.5 | 100 | 7 | −18 | 19 | Light | Y |
| 4 | .01 | 1 | 68.5 | 100 | 7 | 36 | 15.3 | Light | N |
| 5 | .01 | 1 | 68.5 | 100 | 7 | −36 | 15.3 | Light | N |
| 6 | .004 | 2 | 60 | 107.95 | 6.35 | 0 | 50 | Heavy | Y |
| 7 | .004 | 2 | 63 | 107.95 | 6.35 | 36 | 34.7 | Heavy | Y |
| 8 | .004 | 2 | 66.5 | 107.95 | 6.35 | 36 | 30.25 | Heavy | Y |
| 9 | .007 | 2 | 66.5 | 107.95 | 6.35 | 36 | 42.3 | Heavy | Y |
| 10 | .007 | 2 | 69 | 107.95 | 6.35 | 45 | 33 | Heavy | Y |
| 11 | .007 | 2 | 62.4 | 101.6 | 6.35 | 45 | 25.5 | Heavy | Y |
| 12 | .007 | 2 | 60.2 | 101.6 | 6.35 | 45 | 31.25 | Heavy | Y |
| 13 | .007 | 2 | 60.2 | 95.25 | 6.35 | 45 | 16 | Heavy | N |
| 14 | .007 | 2 | 56.75 | 95.25 | 6.35 | 45 | 23.75 | Heavy | Y |
| 15 | .007 | 2 | 57.5 | 95.25 | 6.35 | 54 | 20.1 | Heavy | N |
| 16 | .007 | 2 | 56.75 | 95.25 | 6.35 | 45 | 29 | Heavy | Y |
| 17 | .007 | 2 | 52.5 | 88.9 | 6.35 | 45 | 23.25 | Heavy | Y |
| 18 | .007 | 2 | 55.75 | 88.9 | 6.35 | 54 | 16.6 | Heavy | N |
| 19 | .007 | 2 | 50.25 | 88.9 | 6.35 | 45 | 25.3 | Heavy | Y |

During testing, various jig designs were tested using the testing jig and modifying the various parameters discussed above. Force was assessed qualitatively. More specifically, a dense projectile was positioned in front of the sled in its tensioned position as shown in FIG. 19B, and the sled 314 was urged distally toward the projectile, thereby firing the projectile toward the elongate member 306A. The resulting impact caused by the forceful collision of the projectile into the elongate member 306A was observed and the firing force was thereby categorized as “light” or “heavy” based on these observations. If the resulting impact caused the projectile to travel a distance less than three inches, it was categorized as “light.” If the resulting impact caused the projectile to travel a distance greater than three inches and less than one foot, it was categorized as “medium.” If the resulting impact caused the projectile to travel a distance greater than one foot, it was categorized as “heavy.”

Force and displacement are coupled such that parameter adjustment to achieve a desired displacement can result in loss of force. One way to avoid loss of force is to stack or layer the flexures as discussed above. As such, a desired amount of displacement was achieved via testing, and then that specific flexure design was used in multiples of at least two or more via stacking to achieve the desired amount of force.

Although the various embodiments have been described with reference to preferred implementations, persons skilled in the art will recognize that changes may be made in form and detail without departing from the spirit and scope thereof.

What is claimed is:

1. A biopsy device comprising:
(a) a body;
(b) a biopsy needle extending from the body, the biopsy needle comprising:
(i) an outer cannula; and
(ii) an inner needle slidably disposed within the outer cannula;
(c) an actuation mechanism disposed within the body and operably coupled to the outer cannula such that outer cannula is axially constrained in relation to the actuation mechanism, the actuation mechanism comprising at least one tensionable member, wherein the at least one tensionable member is moveable between a first member position and a second member position;
(d) an actuation handle operably coupled to the inner needle such that the inner needle is axially constrained in relation to the actuation mechanism, wherein the outer cannula is slidably coupled to the actuation handle, wherein the actuation handle is moveable between a first handle position, a second handle position, and an intermediate handle position,
wherein movement of the handle from the intermediate handle position toward the first handle position causes the actuation mechanism to urge the outer cannula distally.

2. The biopsy device of claim 1, wherein when the actuation handle is in the first handle position and the at least one tensionable member is in the first member position, the outer cannula and the inner needle are disposed in an extended position.

3. The biopsy device of claim 1, wherein when the actuation handle is in the second handle position and the at least one tensionable member is in the second member position, the outer cannula and the inner needle are disposed in a retracted position.

4. The biopsy device of claim 1, wherein when the actuation handle is in the intermediate handle position and the at least one tensionable member is in the second member position, the outer cannula and the inner needle are disposed in a tissue collection position, wherein a distal end of the inner needle extends from the outer cannula such that a tissue collection channel on the inner needle is exposed.

5. The biopsy device of claim 1, wherein the outer cannula is urged distally at a speed ranging from about 1.5 m/s to about 20 m/s and a force ranging from about 15 N to about 75 N.

6. The biopsy device of claim 1, wherein the at least one tensionable member is attached at a first end to a first side wall of the body and at a second end to a second side wall, wherein the at least one tensionable member is spaced from a top wall and a bottom wall of the body.

7. A biopsy device comprising:
(a) a body;
(b) a biopsy needle extending from a distal end of the body, the biopsy needle comprising:
(i) an outer cannula; and
(ii) an inner needle slidably disposed within the outer cannula;
(c) an actuation handle extending from a proximal end of the body and coupled to the inner needle, the actuation handle comprising:
(i) an elongate handle body moveably disposed at least partially within the body; and
(ii) a piston cavity defined within the elongate body;
wherein the actuation handle is moveable between a distal handle position, a proximal handle position, and an intermediate handle position; and
(d) an actuation mechanism disposed within the body, the actuation mechanism comprising:
(i) a cannula piston moveably disposed within the piston cavity and coupled to the outer cannula; and
(ii) at least one tensionable member coupled to the cannula piston, wherein the tensionable member is movable between a distal member position and a proximal member position.

8. The biopsy device of claim 7, wherein movement of the handle from the intermediate handle position toward the distal handle position causes the cannula piston to move distally, whereby the at least one tensionable member becomes tensioned and thereby urges the outer cannula distally.

9. The biopsy device of claim 8, wherein the outer cannula is urged distally at a speed ranging from about 1.5 m/s to about 20 m/s and a force ranging from about 15 N to about 75 N.

10. The biopsy device of claim 7, wherein
the outer cannula comprises a cannula coupling body at a proximal end of the outer cannula, and
the cannula piston comprises a cannula cavity defined with the cannula piston, wherein the cannula cavity is sized and shaped to receive the cannula coupling body.

11. The biopsy device of claim 7, wherein
the inner needle comprises a needle coupling body at a proximal end of the inner needle, and
the actuation handle comprises a needle cavity defined within elongate handle body, wherein the needle cavity is sized and shaped to receive the needle coupling body.

12. The biopsy device of claim 7, wherein the at least one tensionable member comprises a at least one distal tensionable member and at least one proximal tensionable member.

13. The biopsy device of claim 12, wherein the at least one distal tensionable member comprises at least two distal tensionable members coupled together and the at least one proximal tensionable member comprises at least two proximal tensionable members coupled together.

14. The biopsy device of claim 7, wherein the at least one tensionable member is attached at a first end to a first side wall of the body and at a second end to a second side wall of the body, wherein the at least one tensionable member is spaced from a top wall and a bottom wall of the body such that the at least one tensionable member is not in contact with the top wall or the bottom wall.

15. The biopsy device of claim 7, wherein the cannula piston is spaced from a top wall of the piston cavity and from a bottom wall of the piston cavity such that the cannula piston is not in contact with the top wall of the piston cavity or the bottom wall of the piston cavity.

16. The biopsy device of claim 7, wherein the at least one tensionable member is a bistable tensionable member, wherein the at least one tensionable member is untensioned in both the distal member position and the proximal member position.

17. The biopsy device of claim 7, further comprising an elongate biopsy needle opening defined in a top portion of the body, wherein the biopsy needle opening is sized and shaped to allow a biopsy needle to pass through the biopsy needle opening laterally.

18. A method of collecting tissue from a patient, the method comprising:
retracting an actuation handle of a biopsy device into a retracted position, the biopsy device comprising:
(a) a body;
(b) a biopsy needle extending from the body, the biopsy needle comprising:
(i) an outer cannula; and
(ii) an inner needle slidably disposed within the outer cannula;
(c) an actuation mechanism disposed within the body and operably coupled to the outer cannula such that outer cannula is axially constrained in relation to the actuation mechanism, the actuation mechanism comprising at least one tensionable member disposed in a proximal position; and
(d) the actuation handle operably coupled to the inner needle such that the inner needle is axially constrained in relation to the actuation mechanism, wherein the outer cannula is slidably coupled to the actuation handle;
inserting the biopsy needle into a patient such that a distal tip of the biopsy needle is disposed adjacent to a target tissue;
urging the actuation handle distally into a tissue collection position, whereby the inner needle is urged distally such that a tissue collection channel on the inner needle extends out of the outer cannula; and
urging the actuation handle distally from the tissue collection position such that the at least one tensionable member becomes tensioned and urges the outer cannula distally over the tissue collection channel, thereby severing tissue disposed in the tissue collection channel.

19. The method of claim 17, wherein the urging the actuation handle distally into a tissue collection position does not move the actuation mechanism.

20. The method of claim 17, wherein the urging the actuation handle distally from the tissue collection position causes the actuation mechanism to be urged distally.
